(19) United States
(12) Patent Application Publication
(10) Pub. No.: US 2020/0066411 A1
(43) Pub. Date: Feb. 27, 2020

(54) CONCEPT UNIQUE IDENTIFIER HINTING FOR HEALTH CARE VALUE SET CURATION

(71) Applicant: CERNER INNOVATION, INC., Kansas City, KS (US)

(72) Inventors: JAMES GRITTER, PARKVILLE, MO (US); DAVID PEKRUL, SHAWNEE, KS (US); DEVIN CRAFT, OVERLAND PARK, KS (US)

(21) Appl. No.: 16/108,982

(22) Filed: Aug. 22, 2018

(51) Int. Cl. G16H 70/20; G16H 40/20; G06F 17/30

(52) U.S. Cl. CPC G16H 70/20; G06F 17/30994; G16H 40/20

(57) ABSTRACT

Embodiments of the present disclosure relate to systems, methods, and user interfaces for providing CUI hinting for health care value set curation. Initially, a code corresponding to a CUI is received. The state of each CUI for a value set is established based on the current included and excluded codes corresponding to each CUI. Codes may have more than one CUI assigned, but the hinting process may only use codes with a single assigned CUI. Based on the state of each CUI for the value set, a hint is provided for the code. The hint is based on the state of the CUI and may prompt a user to include, exclude, or review the code for the particular value set or inform the user that the code is inherently ambiguous with respect to the particular value set. The CUI hinting provides higher quality, consistency, and efficiency during the curation process.

### BACKGROUND

There are many different code vocabularies in use in health care today. When aggregating data across data sources, cross-vocabulary value sets can be employed to group similar codes together. These value sets often group many fine-grained codes into a higher-level concept as well. In order to query for patients with a diagnosis of diabetes, a terminologist builds a value set of codes that represent diabetes generically, as well as all the codes for more specific variants of diabetes. Such a value set might have thousands of codes across multiple vocabularies and at different granularities. Across the entire health care spectrum, there are thousands of such value sets that represent many diagnoses. The curation and maintenance tasks can be overwhelming. Each vocabulary releases new codes periodically with varying intervals, resulting in a constant influx of new codes to consider for inclusion in all existing value sets.

Previous systems have attempted to use include or exclude decisions and Unified Medical Language System (UMLS) concept unique identifiers (CUIs) in a straightforward way. However, the straightforward logic of these systems results in imprecise hints. For example, the straightforward logic infers that all CUIs associated with an included or excluded code should be consistent across other inclusion and exclusion decisions. But, that ignores that the decisions may be made based on a single CUI associated with that code, not necessarily all of them. This leads to a percentage of incorrect hints in every category, and that percentage spoils any efficiency gains.

### BRIEF SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present disclosure relate to systems, methods, and user interfaces for providing CUI hinting for health care value set curation. More particularly, embodiments of the present disclosure provides higher quality, consistency, and efficiency during the curation process. Initially, a code corresponding to a CUI is received. Next, the state of each CUI for a value set is established based on the current included and excluded codes corresponding to each CUI. Codes may have more than one CUI assigned to them, but the hinting process may only use codes with a single assigned CUI. Based on the state of each CUI for the value set, a hint is provided for the code. The hint is based on the state of the CUI and may prompt a user to include, exclude, or review the code for the particular value set or inform the user that the code is inherently ambiguous with respect to the particular value set.

### BRIEF DESCRIPTION OF THE SEVERAL VIEWS OF THE DRAWINGS

The patent or application file contains at least one drawing executed in color. The present invention is described in detail below with reference to the attached drawing figures, wherein:

FIG. 1 is a block diagram of an exemplary operating environment suitable to implement embodiments of the present invention;

FIG. 2 depicts an exemplary framework of a CUI hinting system suitable to implement embodiments of the present invention;

FIG. 3 depicts a flow diagram illustrating concept unique identifier hinting for undecided codes, in accordance with an embodiment of the present invention;

FIG. 4 depicts a flow diagram illustrating concept unique identifier hinting for included and excluded codes, in accordance with an embodiment of the present invention;

FIG. 5 depicts a flow diagram illustrating concept unique identifier hinting for undecided codes in a template use case, in accordance with an embodiment of the present invention;

FIG. 6 depicts a flow diagram illustrating concept unique identifier hinting for included and excluded codes in a template use case, in accordance with an embodiment of the present invention;

FIG. 7 depicts an illustrative screen display of concept unique identifier hinting for undecided codes in an abnormal pulse template use case, in accordance with embodiments of the present invention;

FIG. 8 depicts an illustrative screen display concept unique identifier hinting for undecided codes in an acyclovir ophthalmic template use case, in accordance with embodiments of the present invention;

FIG. 9 depicts a flow diagram of a method for concept unique identifier hinting for undecided codes, in accordance with an embodiment of the present invention; and

FIG. 10 is a flow diagram of a method for concept unique identifier hinting for included and excluded codes, in accordance with embodiments of the invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” might be used herein to connote different elements of methods employed, the terms should not be interpreted as implying any particular order among or between various steps herein disclosed unless and except when the order of individual steps is explicitly stated.

As noted in the background, there are many different code vocabularies in use in health care today. When aggregating data across data sources, cross-vocabulary value sets can be employed to group similar codes together. These value sets often group many fine-grained codes into a higher-level concept as well. In order to query for patients with a diagnosis of diabetes, a terminologist builds a value set of codes that represent diabetes generically, as well as all the codes for more specific variants of diabetes. Such a value set might have thousands of codes across multiple vocabularies and at different granularities. Across the entire health care spectrum, there are thousands of such value sets that represent many diagnoses. The curation and maintenance tasks can be overwhelming. Each vocabulary releases new codes periodically with varying intervals, resulting in a constant influx of new codes to consider for inclusion in all existing value sets.

Previous systems have attempted to use include or exclude decisions and Unified Medical Language System (UMLS) concept unique identifiers (CUIs) in a straightforward way. However, the straightforward logic of these systems results in imprecise hints. For example, the straightforward logic infers that all CUIs associated with an included or excluded code should be consistent across other inclusion and exclusion decisions. But, that ignores that the decisions may be made based on a single CUI associated with that code, not necessarily all of them. This leads to a percentage of incorrect hints in every category, and that percentage spoils any efficiency gains.

Although this disclosure utilizes the term CUI (such as a CUI for code displays provided by the Unified Medical Language System (UMLS), it is contemplated that other CUI types (such as RXCUIS for code displays provided by RxNorm, a drug database created by the National Library of Medicine (NLM)) are within the scope of the present invention.

Embodiments of the present disclosure relate to systems, methods, and user interfaces for providing CUI hinting for health care value set curation. More particularly, embodiments of the present disclosure provides higher quality, consistency, and efficiency during the curation process. Initially, a code corresponding to a CUI is received. Next, the state of each CUI for a value set is established based on the current included and excluded codes corresponding to each CUI. Codes may have more than one CUI assigned to them, but the hinting process may only use codes with a single assigned CUI. Based on the state of each CUI for the value set, a hint is provided for the code. The hint is based on the state of the CUI and may prompt a user to include, exclude, or review the code for the particular value set or inform the user that the code is inherently ambiguous with respect to the particular value set.

In embodiments, if all the decided single CUI codes for a given CUI are included in the value set, then the CUI is a definitive yes. If all the decided single CUI codes for a given CUI are excluded in the value set, then the CUI is a definitive no. If there is at least one single CUI code included and at least one single CUI code excluded for a given CUI, then the CUI is inconsistent.

While only single CUI codes may be used to assign CUI state, all codes with one or more CUIs are candidates to be hinted. If all CUIs on a code are a definitive yes and the code is undecided, it will be hinted to be included. If all CUIs on a code are a definitive yes and the code is excluded, it will be hinted that it needs review. The same pattern exists if all CUIs on a code are a definitive no. Any decided single CUI code with an inconsistent CUI will be hinted to review. A code with at least one definitive yes CUI and at least one definitive no CUI will be hinted as ambiguous.

For clarity, the above hinting logic applies if the use case for the value set is known. There is a different set of hinting rules for a special case of value set referred to as a template value set or template. A template can be used to create other value sets, so if there is any doubt about a code it will be included. This results in a slightly different hinting logic, described below.

For example, if any of the CUIs on a code are a definitive yes and the code is undecided, it will hint to include. If it is excluded, the code is hinted to review. If all of the CUIs on a code are a definitive no and the code is undecided, the code is hinted to exclude. If it is included, the code is hinted to review. The codes hinted as ambiguous above (at least one definitive yes CUI and at least one definitive no CUI) are hinted to include for template value sets.

Accordingly, one embodiment of the present disclosure is directed to one or more computer storage media having computer-executable instructions embodied thereon that, when executed by a computer, causes the computer to perform operations for providing CUI hinting for health care value set curation. The operations include identifying an undecided code corresponding to a CUI. The operations also include determining a state of a CUI for a particular value set, the state indicated by included codes, excluded codes, and ambiguous codes corresponding to the CUI with respect to the particular value set. The operations further include providing a hint for the undecided code, via a user interface, the hint based on the state of the CUI and prompting a user to include, exclude, or review the undecided code for the particular value set.

In another embodiment, the present disclosure directed to a computerized method providing CUI hinting for health care value set curation. The method comprises identifying an included or excluded code having a CUI for a particular value set. The method also comprises determining a state of the CUI for the particular value set, the state indicated by included codes, excluded codes, and ambiguous codes corresponding to the CUI with respect to the particular value set. The method further comprises providing a hint to a user, via a user interface, the hint based on the state of the CUI and prompting the user to accept or review the included or excluded code for the particular value set.

In yet another embodiment, the present disclosure is directed to a system for providing CUI hinting for health care value set curation. The system comprises a processor; and a computer storage medium storing computer-usable instructions that, when used by the processor, cause the processor to: identify a code corresponding to a CUI; determine a state of the CUI for a particular value set, the state indicated by included codes, excluded codes, and ambiguous codes corresponding to the CUI with respect to the particular value set; and provide a hint for the code, via a user interface, the hint based on the state of the CUI and prompting a user to include, exclude, or review the code for the particular value set or inform the user that the code is inherently ambiguous with respect to the particular value set.

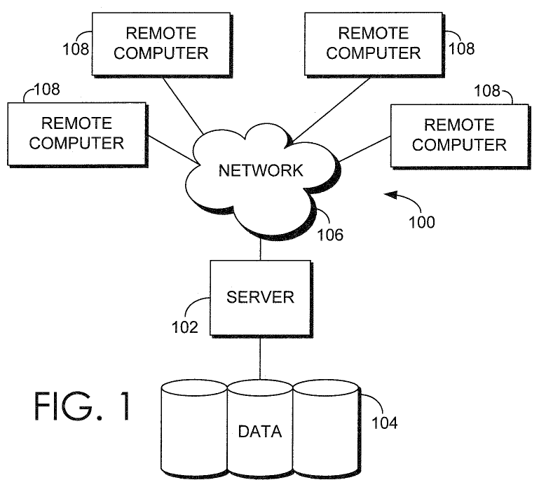

Having briefly described embodiments of the present invention, an exemplary operating environment suitable for use in implementing embodiments of the present invention is described below. FIG. 1 provides an aspect of an example operating environment with which embodiments of the present invention may be implemented. The aspect of an operating environment is illustrated and designated generally as reference numeral 100.

Example operating environment 100 comprises a general purpose computing device in the form of a control server 102. Exemplary components of the control server 102 comprise a processing unit, internal system memory, and a suitable system bus for coupling various system components, including database cluster 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

Control server 102 typically includes therein, or has access to, a variety of computer-readable media, for instance, database cluster 104. Computer-readable media can be any available media that might be accessed by control server 102, and includes volatile and nonvolatile media, as well as, removable and nonremovable media. Computer-readable media might include computer storage media. Computer storage media includes volatile and nonvolatile media, as well as removable and nonremovable media implemented in any method or technology for storage of information, such as computer-readable instructions, data structures, program modules, or other data. In this regard, computer storage media might comprise RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the control server 102. Computer storage media does not comprise signals per se. Combinations of any of the above also may be included within the scope of computer-readable media.

The computer storage media discussed above and illustrated in FIG. 1, including database cluster 104, provide storage of computer-readable instructions, data structures, program modules, and other data for the control server 102. In some embodiments, data cluster 104 takes the form of a cloud-based data store, and in some embodiments is accessible by a cloud-based computing platform.

The control server 102 might operate in a computer network 106 using logical connections to one or more remote computers 108. Remote computers 108 might be located at a variety of locations in a medical or research environment, including clinical laboratories (e.g., molecular diagnostic laboratories), hospitals and other inpatient settings, veterinary environments, ambulatory settings, medical billing and financial offices, hospital administration settings, home health care environments, and providers' offices. Providers may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like.

The remote computers 108 might also be physically located in nontraditional medical care environments so that the entire health care community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Exemplary computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networked environment, program modules or portions thereof might be stored in association with the control server 102, the database cluster 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a pointing device (commonly referred to as a mouse), a trackball, or a touch pad. Other input devices comprise microphones, satellite dishes, scanners, or the like. Commands and information might also be sent directly from a remote health care device to the control server 102. In addition to a monitor, the control server 102 and/or remote computers 108 might comprise other peripheral output devices, such as speakers and a printer.

In some embodiments, control server 102 is a computing system or platform made up of one or more computing devices. Embodiments of control server 102 may be a distributed computing system, a centralized computing system, a single computer such as a desktop or laptop computer or a networked computing system. Thus, in some embodiments, control server 102 comprises a multi-agent computer system with software agents.

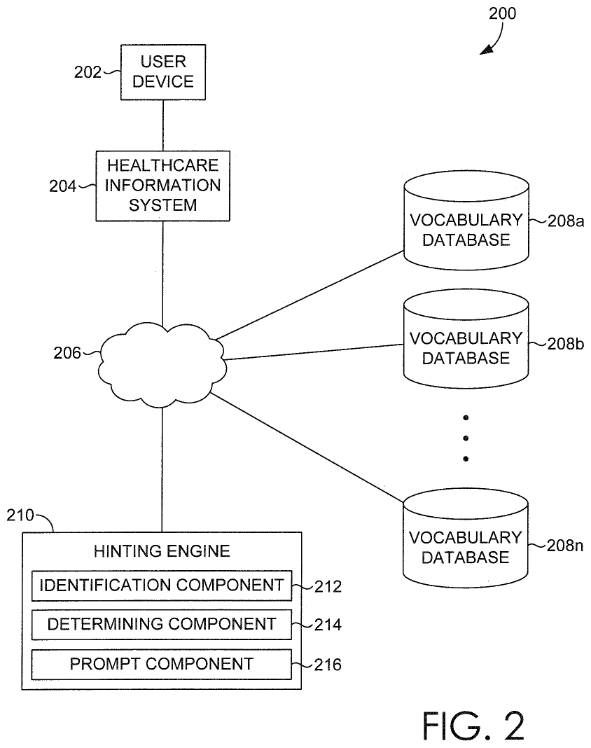

Turning now to FIG. 2, an exemplary framework of a CUI hinting system 200 is shown, in accordance with an aspect of the present invention. It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, various functions may be carried out by a processor executing instructions stored in memory. The CUI hinting system 200 may be implemented via any type of computing device, such as computing device 100 described above with reference to FIG. 1, for example.

The CUI hinting system 200 generally operates to provide CUI hinting for health care value set curation. More particularly, the CUI hinting system 200 provides higher quality, consistency, and efficiency during the curation process. To do so, the CUI hinting system initially receives a code corresponding to a CUI. Next, the state of each CUI for a value set is established by the CUI hinting system 200 based on the current included and excluded codes corresponding to each CUI. Codes may have more than one CUI assigned to them, but the hinting process may only use codes with a single assigned CUI. Based on the state of each CUI for the value set, a hint is provided by the CUI hinting system 200 for the code. The hint is based on the state of the CUI and may prompt a user to include, exclude, or review the code for the particular value set or inform the user that the code is inherently ambiguous with respect to the particular value set.

As shown in FIG. 2, the CUI hinting system 200 includes, among other components not shown, user device 202, health care information system 204, vocabulary databases 208a -208n and, a hinting engine 210. It should be understood that the CUI hinting system 200 shown in FIG. 2 is an example of one suitable computing system architecture. Each of the components shown in FIG. 2 may be implemented via any type of computing device, such as computing device 100 described with reference to FIG. 1, for example.

The components may communicate with each other via a network 206, which may include, without limitation, one or more local area networks (LANs) and/or wide area networks (WANs). Such networking environments are commonplace in offices, enterprise-wide computer networks, intranets, and the Internet. It should be understood that any number of hinting engines and vocabulary databases may be employed within the CUI hinting system 200 within the scope of the present disclosure. Each may comprise a single device or multiple devices cooperating in a distributed environment. For instance, the hinting engine 210 (or any of its components: identification component 212, a determination component 214, and a prompt component 216) and/or the vocabulary databases 208a -208n may be provided via multiple devices arranged in a distributed environment that collectively provide the functionality described herein. In other embodiments, a single device may provide the functionality of multiple components of the CUI hinting system 200. For example, a single device may provide the hinting engine 210 and/or the vocabulary databases 208a -208n. In some embodiments, some or all functionality provided by the hinting engine 210 (or any of its components) and/or the vocabulary databases 208a -208n may be provided by a user device. Additionally, other components not shown may also be included within the network environment.

Generally, the hinting engine 210 provides CUI hinting for health care value set curation. The CUI hinting provided by hinting engine 210 provides higher quality, consistency, and efficiency during the curation process. In embodiments, a user may access and utilize the features of the hinting engine 210 via a health care information system 204.

As described above, the hinting engine 210 includes several components including an identification component 212, a determination component 214, and a prompt component 216. The identification component 212 generally receives codes corresponding to a CUI. The codes may be received from vocabulary database 208a -208n.

The determination component 214 generally determines the state of the CUI for a value set based on the current included and excluded codes corresponding to the CUI. In a first example, when the use case for the value set is known, if all the decided single CUI codes for a given CUI are included in the value set, then the determination component 214 determines the CUI is a definitive yes. If all the decided single CUI codes for a given CUI are excluded in the value set, then the determination component 214 determines the CUI is a definitive no. If there is at least one single CUI code included and at least one single CUI code excluded for a given CUI, then the determination component 214 determines the CUI is inconsistent.

The prompt component 216 generally provides a hint, via a user interface of the user device 202. The hint may prompt a user to include, exclude, or review the code for the particular value set or inform the user that the code is inherently ambiguous with respect to the particular value set. Continuing the first example, while only single CUI codes may be used to assign CUI state, all codes with one or more CUIs are candidates to be hinted by the prompt component 216. If all CUIs on a code are a definitive yes and the code is undecided, the prompt component 216 hints the code to be included. If all CUIs on a code are a definitive yes and the code is excluded, the prompt component 216 hints the code needs review. The same pattern exists if all CUIs on a code are a definitive no. Any decided single CUI code with an inconsistent CUI will be hinted by the prompt component 216 to review. A code with at least one definitive yes CUI and at least one definitive no CUI will be hinted by the prompt component 216 as ambiguous.

In a second example, a template value set use case, if there is any doubt about a code, the prompt component 216 hints the code to be included. For example, if any of the CUIs on a code are a definitive yes and the code is undecided, the prompt component 216 hints to include the code. If it is excluded, the prompt component 216 hints to review the code. If all of the CUIs on a code are a definitive no and the code is undecided, the prompt component 216 hints to exclude the code. If it is included, the prompt component 216 hints to review the code. For the codes hinted as ambiguous above (at least one definitive yes CUI and at least one definitive no CUI) the prompt component 216 hints to include the code for template value sets.

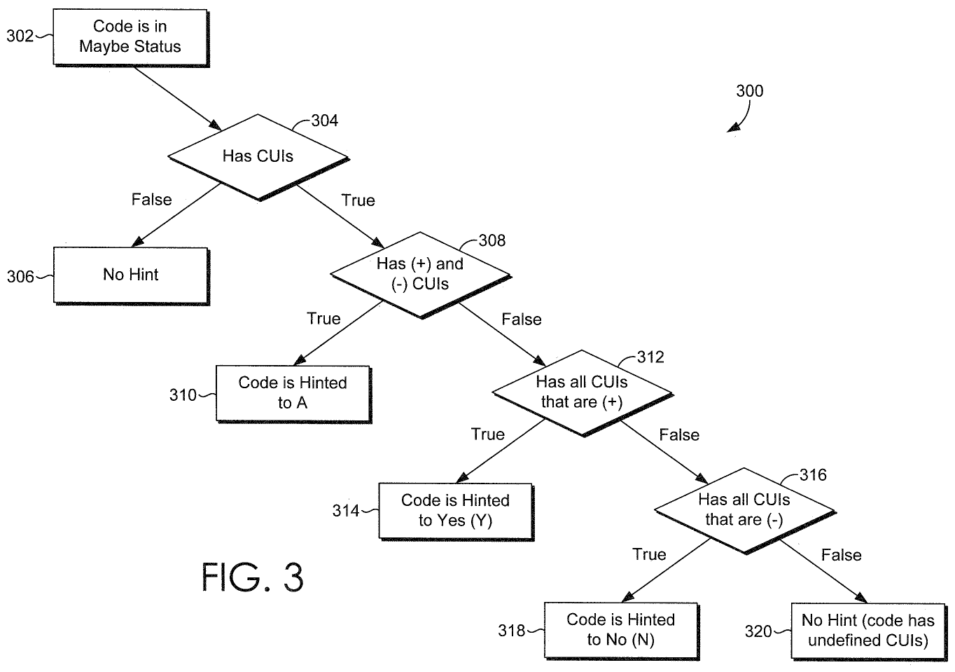

Referring now to FIG. 3, an exemplary flow diagram 300 illustrates concept unique identifier hinting for undecided codes, in accordance with an embodiment of the present invention.

Initially, as shown at step 302, a code is received in an undecided or maybe status. It is determined whether the code has CUIs, at step 304. If it does not have CUIs, no hint is provided, at step 306. If the code has CUIs, the state of the CUIs is determined. If the code has CUIs that are both included and excluded, as shown at step 308, the code is hinted as ambiguous, at step 310, and needs additional review. If the code has all CUIs that are included, as shown at step 312, the code is hinted to include, at step 314. If on the other hand, the code has all CUIs that are excluded, as shown at step 316, the code is hinted to exclude, at step 318. However, if the code has undefined CUIs, no hint is provided, at step 320.

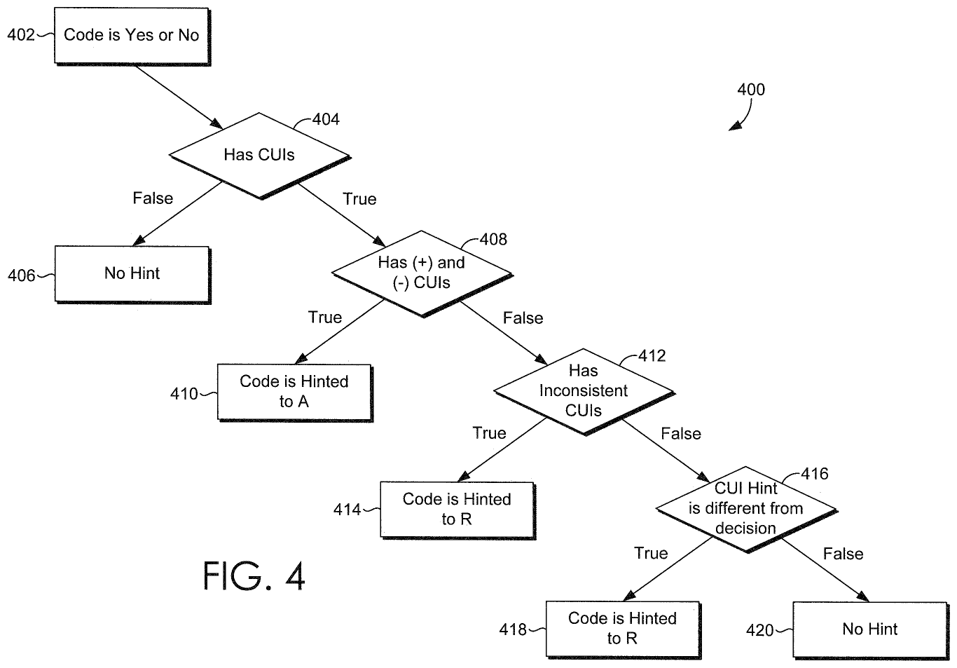

In FIG. 4, an exemplary flow diagram 400 illustrates concept unique identifier hinting for included and excluded codes, in accordance with an embodiment of the present invention.

Initially, as shown at step 402, a code is received in an included or excluded status. It is determined whether the code has CUIs, at step 404. If it does not have CUIs, no hint is provided, at step 406. If the code has CUIs, the state of the CUIs is determined. If the code has CUIs that are both included and excluded, as shown at step 408, the code is hinted as ambiguous, at step 410, and needs additional review. If the code does not have CUIs that are included and excluded but has inconsistent CUIs, as shown at step 412, the code is hinted to review, at step 414. If the code does not have CUIs that are included and excluded, does not have inconsistent CUIs, but the CUI hint is different from the decision, as shown at step 416, the code is hinted to review, at step 418. If the code does not have CUIs that are included and excluded, does not have inconsistent CUIs, and the CUI hint is not different from the decision, as shown at step 418, no hint is provided, as shown at step 420.

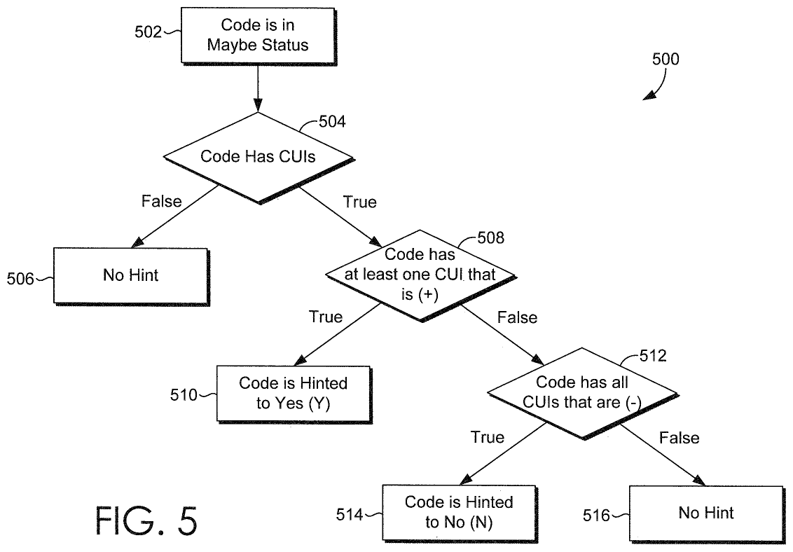

Referring now to FIG. 5, an exemplary flow diagram 500 illustrates concept unique identifier hinting for undecided codes in a template use case, in accordance with an embodiment of the present invention.

Initially, as shown at step 502, a code is received in an undecided or maybe status. It is determined whether the code has CUIs, at step 504. If it does not have CUIs, no hint is provided, at step 506. If the code has CUIs, the state of the CUIs is determined. If the code has at least one CUI that is included, as shown at step 508, the code is hinted to include, at step 510. If the code has all CUIs that are excluded, as shown at step 512, the code is hinted to exclude, at step 514. However, if the code has all undefined CUIs, no hint is provided, at step 516.

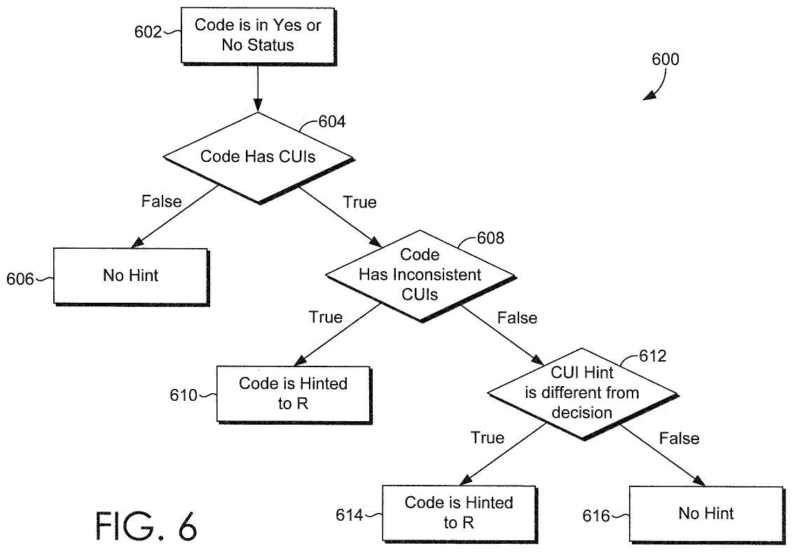

In FIG. 6, an exemplary flow diagram 600 illustrates concept unique identifier hinting for included and excluded codes in a template use case, in accordance with an embodiment of the present invention.

Initially, as shown at step 602, a code is received in an included or excluded status. It is determined whether the code has CUIs, at step 604. If it does not have CUIs, no hint is provided, at step 606. If the code has CUIs, the state of the CUIs is determined. If the code has CUIs that are inconsistent, as shown at step 508, the code is hinted to review, at step 610. If the code does not have CUIs that inconsistent CUIs, but the CUI hint is different from the decision, as shown at step 612, the code is hinted to review, at step 614. If the code does not have CUIs that are inconsistent, and the CUI hint is not different from the decision, as shown at step 616, no hint is provided.

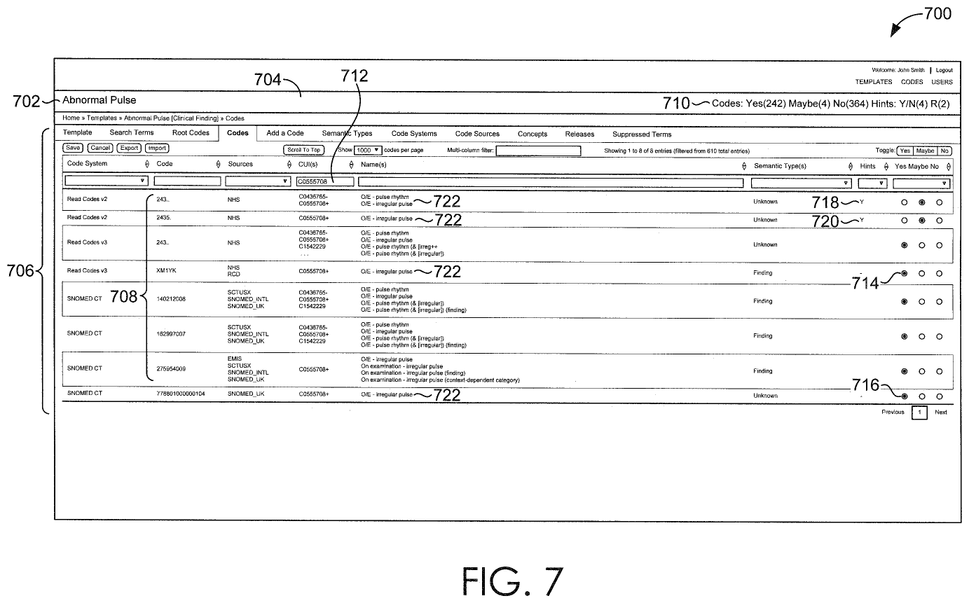

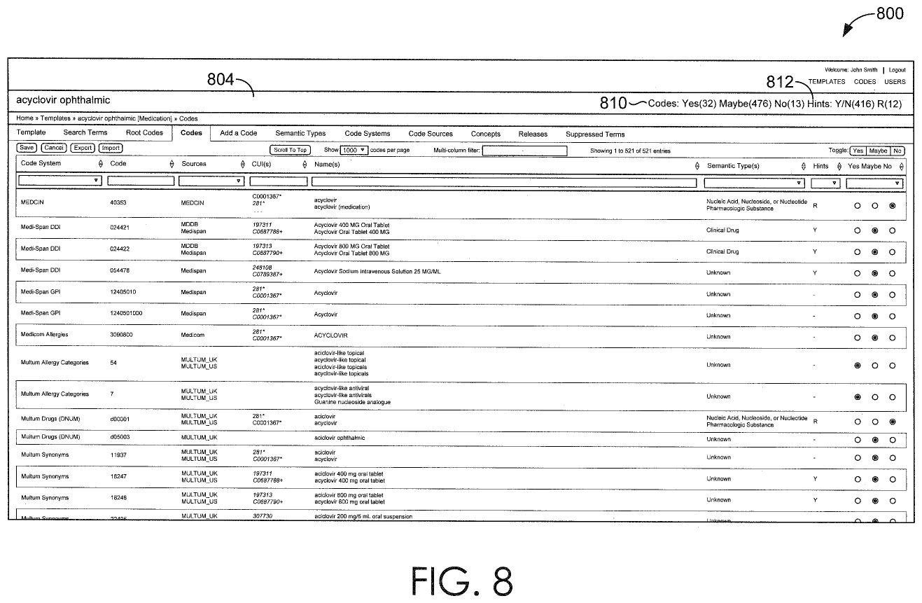

With reference to FIGS. 7-8, illustrative screen displays 700 and 800 of embodiments of the present invention are shown. It is understood that each of the illustrative screen displays are connected logically, such that they comprise a user interface designed for providing CUI hinting for health care value set curation. The screen displays may appear in any order and with any number of screen displays, without regard to whether the screen display is described or depicted herein. The screen displays provide tools that enable higher quality, consistency, and efficiency during the curation process, in accordance with embodiments of the present invention.

Referring initially, to FIG. 7, an illustrative screen display 700 of concept unique identifier hinting for undecided codes in an abnormal pulse template use case is depicted, in accordance with embodiments of the present invention. The name of the template 702 may be provided in the top banner 704. The table 706 contains the codes 708 that are being considered for inclusion in the template. As can be appreciated, there may be more codes in the template than are shown in the display 700. On the right side of the top banner 704, a number of code counts 710 that have been included (the yes count), excluded (the no count), and still need to be decided (the maybe count) are provided.

As illustrated, only eight codes are currently visible in the display 700 because the template has been filtered to show codes that have been assigned the CUI of “C0555708” 712. As illustrated, a simple yes hint has been provided because the user or content author has already marked the “XM1YK” 714 and “778801000000104” 716 codes as yes (include), so the CUI hinting system hints 718, 720 that “243..” and “2435.” should also be included in the template. More clearly, because each of the codes (“XM1YK,” “778801000000104,” “243 . . . ,” and “2435.”) have all been assigned the C0555708 CUI, the systems hints that “243. . . ” and “2435.” should be included in the template. In this example, the CUI assignment came from each of the codes having “OLE—irregular pulse” as a display. However, as synonyms may share the same CUI, it should be noted the displays do not have to be exactly the same.

In this example, “243 . . . ” is also hinted to yes or include despite the fact that “243 . . . ” also has a display of “OLE—pulse rhythm.” Pulse rhythm is not an abnormal pulse, but the system still hints to include “243 . . . ” because templates are intentionally biased to include if there is any doubt. Additionally, illustrative screen display 700 shows that despite saying yes to “243 . . . ,” “'140212008,” and 162997007” (which all have the C0436765 CUI aligning with “O/E—pulse rhythm”), the system recognizes that it does not mean other codes with C0436765 should be hinted to yes. That is evidenced by the minus sign appended to the CUI.

In FIG. 8, an illustrative screen display 800 of concept unique identifier hinting for undecided codes in an acyclovir ophthalmic template use case is depicted, in accordance with embodiments of the present invention. In this example, similar to FIG. 7, the counts in the top banner 804 illustrate the number of code counts 810 that have been included (the yes count), excluded (the no count), and still need to be decided (the maybe count) are provided. Additionally, the code counts also illustrate the number hints that have been provided. For example, in this case, there are thirty two included and thirteen excluded codes. There are also four hundred seventy-six that still need to be decided. However, of those four hundred seventy-six, four hundred sixteen have either an included or excluded hint. Because of these hints that have a high probability of accuracy, those four hundred seventy-six codes can be can be quickly confirmed by toggling the in batch to included or excluded. In other words, even though the terminologist has just begun to work through defining this template, the hinting process has significantly reduced the work to do so.

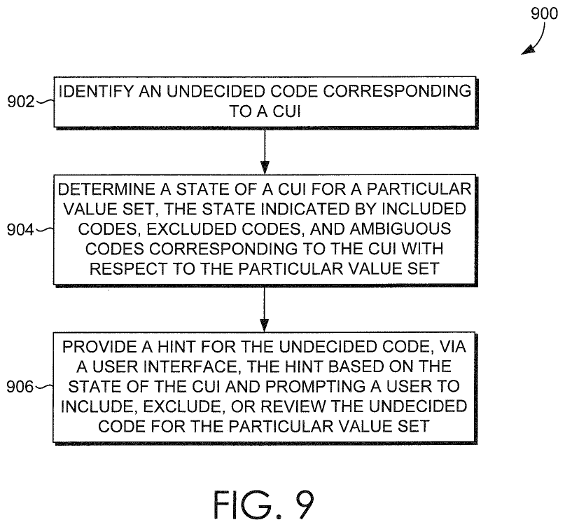

Turning now to FIG. 9, a flow diagram is provided illustrating a method 500 for concept unique identifier hinting for undecided codes, in accordance with embodiments of the present invention. Method 900 may be performed by any computing device (such as computing device described with respect to FIG. 1) with access to an CUI hinting system (such as the one described with respect to FIGS. 2 and 4) or by one or more components of the CUI hinting system.

Initially, at step 902, an undecided code corresponding to a CUI is identified.

At step 904, a state of a CUI for a particular value set is determined. The state may be indicated by included codes, excluded codes, and ambiguous codes corresponding to the CUI with respect to the particular value set.

At step 906, a hint is provided, via a user interface, for the undecided code. The hint is based on the state of the CUI and prompts a user to include, exclude, or review the undecided code for the particular value set.

In some embodiments, the state of the CUI for the particular value set is determined to be positive or included. In one aspect, the state of the CUI being positive indicates all codes corresponding to the CUI are included codes. In another aspect, the state of the CUI being positive indicates at least one code corresponding to the CUI is an included code. In either case, the hint may prompt the user to include the undecided code for the particular value set.

In some embodiments, the state of the CUI for the particular value set is determined to be negative. The state of the CUI being negative indicates all codes corresponding to the CUI are excluded codes. In this case, the hint prompts the user to exclude the undecided code for the particular value set.

In some embodiments, the state of the CUI for the particular value set is determined to be ambiguous. In this case, the hint prompts the user to review the undecided code for the particular value set.

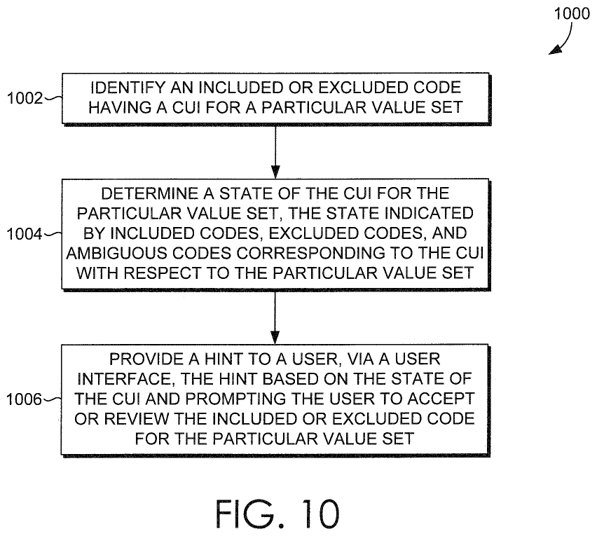

Turning now to FIG. 10, a flow diagram is provided illustrating a method 1000 for concept unique identifier hinting for included and excluded codes, in accordance with embodiments of the present invention. Method 1000 may be performed by any computing device (such as computing device described with respect to FIG. 1) with access to an CUI hinting system (such as the one described with respect to FIGS. 2 and 4) or by one or more components of the CUI hinting system.

Initially, at step 1002, an included or excluded code having a CUI for a particular value set is identified.

At step 1004, a state of the CUI for the particular value set is determined. The state is indicated by included codes, excluded codes, and ambiguous codes corresponding to the CUI with respect to the particular value set.

At step 1006, a hint is provided, via a user interface, to a user. The hint is based on the state of the CUI and may prompt the user to accept or review the included or excluded code for the particular value set.

In embodiments, the state of the CUI for the particular value set is determined to be positive. In this case, if the code is an included code, the hint prompts the user to accept the included code for the particular value set. If on the other hand, the code is an excluded code, the hint prompt the user to review the excluded code for the particular value set.

In embodiments, the state of the CUI for the particular value set is determined to be negative. In this case, if the code is an included code, the hint prompts the user to review the included code for the particular value set. If on the other hand the code is an excluded code, the hint prompts the user to accept the excluded code for the particular value set.

In some embodiments, it is determined the hint is different from a selection made by the user. In this case, the hint prompt the user to review the selection corresponding to the code for the particular value set.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described. Accordingly, the scope of the invention is intended to be limited only by the following claims.

What is claimed is:

1. One or more computer storage media having computer-executable instructions embodied thereon that, when executed by a computer, causes the computer to perform operations for providing concept unique identifier (CUI) hinting for health care value set curation, the operations comprising:
identifying an undecided code corresponding to a CUI;
determining a state of a CUI for a particular value set, the state indicated by included codes, excluded codes, and ambiguous codes corresponding to the CUI with respect to the particular value set; and
providing a hint for the undecided code, via a user interface, the hint based on the state of the CUI and prompting a user to include, exclude, or review the undecided code for the particular value set or inform the user that the code is inherently ambiguous with respect to the particular value set.

2. The media of claim 1, further comprising determining the state of the CUI for the particular value set is positive.

3. The media of claim 2, wherein the hint prompts the user to include the undecided code for the particular value set.

4. The media of claim 2, wherein the state of the CUI being positive indicates all codes corresponding to the CUI are included codes.

5. The media of claim 2, wherein the state of the CUI being positive indicates at least one code corresponding to the CUI is an included code.

6. The media of claim 1, further comprising determining the state of the CUI for the particular value set is negative.

7. The media of claim 6, wherein the hint prompts the user to exclude the undecided code for the particular value set.

8. The media of claim 6, wherein the state of the CUI being negative indicates all codes corresponding to the CUI are excluded codes.

9. The media of claim 1, further comprising determining the state of the CUI for the particular value set is ambiguous.

10. The media of claim 6, wherein the hint prompts the user to review the undecided code for the particular value set.

11. A computerized method for providing concept unique identifier (CUI) hinting for health care value set curation, the method comprising:
identify an included or excluded code having a CUI for a particular value set;
determine a state of the CUI for the particular value set, the state indicated by included codes, excluded codes, and ambiguous codes corresponding to the CUI with respect to the particular value set; and
provide a hint to a user, via a user interface, the hint based on the state of the CUI and prompting the user to accept or review the included or excluded code for the particular value set.

12. The method of claim 11, further comprising determining the state of the CUI for the particular value set is positive.

13. The method of claim 12, wherein the hint prompts the user to accept the included code for the particular value set.

14. The method of claim 12, wherein the hint prompts the user to review the excluded code for the particular value set.

15. The method of claim 11, further comprising determining the state of the CUI for the particular value set is negative.

16. The method of claim 15, wherein the hint prompts the user to review the included code for the particular value set.

17. The method of claim 15, wherein the hint prompts the user to accept the excluded code for the particular value set.

18. The method of claim 11, further comprising determining the hint is different from a selection made by the user.

19. The method of claim 18, wherein the hint prompts the user to review the selection corresponding to the code for the particular value set.

20. A system for providing concept unique identifier (CUI) hinting for health care value set curation, the system comprising:
a processor; and
a computer storage medium storing computer-usable instructions that, when used by the processor, cause the processor to:
identify a code corresponding to a CUT;
determine a state of the CUI for a particular value set, the state indicated by included codes, excluded codes, and ambiguous codes corresponding to the CUI with respect to the particular value set; and
provide a hint for the code, via a user interface, the hint based on the state of the CUI and prompting a user to include, exclude, or review the code for the particular value set or inform the user that the code is inherently ambiguous with respect to the particular value set.
